(19) United States
(12) Patent Application Publication
(10) Pub. No.: US 2024/0027418 A1
(43) Pub. Date: Jan. 25, 2024

(54) Method, computer program, computer system and assembly for the non-destructive determination of the juice content of juice fruits, as well as the use of this assembly for the quality classification of juice fruits

(71) Applicant: Agricola Lusia S.r.l., Lusia (RO) (IT)

(72) Inventors: Daniele Campagnaro, Lusia (RO) (IT); Nicola Modica, Abano Terme (PD) (IT); Carlo Gaetan, Castelfranco Veneto (TV) (IT); Paolo Girardi, Monselice (PD) (IT); Pietro Riello, Padova (PD) (IT)

(21) Appl. No.: 18/256,479

(22) PCT Filed: Nov. 25, 2021
(86) PCT No.: PCT/IB2021/060981

§ 371 Date: Jun. 8, 2023

(30) Foreign Application Priority Data
Dec. 11, 2020 (IT) 102020000030629

(51) Int. Cl. G01N 33/14

(52) U.S. Cl. CPC G01N 33/14

(57) ABSTRACT

A computer-implemented method for the non-destructive determination of the juice content of a juice fruit includes collecting first data related to the estimated volume and the actual weight of the juice fruit; collecting a first plurality of pairs of reference data for the juice fruit; calculating, based on the first data, the density of the juice fruit; and processing, based on the first plurality of pairs of reference data and the calculated density value, a value related to an amount of an estimated juice amount.

### FIELD OF THE INVENTION

The present invention relates to the technical field of agri-food, and it particularly relates to an assembly, a method, a software and a computer system for the non-destructive determination of juice content of juice fruits.

The invention also relates to the use of the aforementioned assembly for the quality classification of juice fruits.

### Definitions

In the present document, the expression “juice fruit” or derivatives is used to indicate any fruit from which juice can be extracted.

In the present document, the expression “juice content” or derivatives is used to indicate the percentage of juice present in the juice fruit with respect to the total weight of the juice fruit.

In the present document, the expression “quality classification” or derivatives referring to a juice fruit is used to indicate the association of a certain characteristic, for example the percentage of juice contained therein, with such juice fruit.

### State of the Art

It is known that agri-food companies need to classify juice fruits, for example citrus fruits, according to the juice content.

From the regulatory point of view, EC Regulation no 1221/2008 provides for a minimum amount of juice for citrus fruits intended for fresh delivery to the consumer. In the case of oranges, this juice content amounts to 30%.

To date, there are destructive methods which provide for the destruction of the fruit to determine the juice content thereof. These methods clearly cannot be implemented on an industrial scale.

On the other hand, apparatuses for the non-destructive measurement of dimensional characteristics of fruits in general by means of image analysis, are known. An example of such apparatuses is known from the document U.S. Pat. No. 5,449,911.

However, such types of apparatuses do not measure the juice content of the fruits.

### SUMMARY OF THE INVENTION

The object of the present invention is to overcome the drawbacks outlined above by providing an assembly, a method and a computer program for the non-destructive determination of the juice content of juice fruits that is highly efficient and cost-effective.

A further object of the present invention is to provide an assembly, a method, a computer program and a computer system for the non-destructive determination of the juice content of juice fruits that allow the minimum waste of the batch of fruits to be measured and of resources used.

A further object of the present invention is to provide an assembly, a method, a computer program and a computer system for the non-destructive determination of the juice content of juice fruits which is extremely easy to implement even on existing processing lines.

These and other objects which will be more apparent hereinafter, are attained by an assembly, a method, a computer program and a computer system for the non-destructive determination of the juice content of juice fruits, as described and/or claimed and/or illustrated herein.

In particular, the invention relates to a computer-implemented method for the non-destructive determination of the juice content of juice fruits, for example citrus fruits, comprising at least the following steps:
collecting first data relating to the estimated volume and the actual weight of said at least one juice fruit;
collecting at least one first plurality of pairs of reference data for said at least one juice fruit, each pair of reference data consisting of a reference density value and a corresponding value relating to the reference amount of juice;
calculating, based on the first data, the density of the at least one juice fruit;
processing, based on the reference data and the calculated density value, a value relating to an estimated amount of juice for the at least one juice fruit, for example the percentage by weight of juice estimated with respect to the weight of the at least one fruit.

Therefore, this will allow to determine the juice content of the juice fruits in a non-destructive manner. As a matter fact, for each fruit this juice content will be determined simply by estimating the volume thereof, for example through an integrated electronic system of video cameras of the known type, capable of determining the shape and size of the fruit, and weighing it, for example by means of a weighing scale.

Therefore, this will allow to obtain the density thereof in a per se known manner and to compare this datum with the reference ones, in which each density value corresponds to a determined average juice content.

The reference data may be data already available and implemented in the computer, for example data relating to juice fruits of a predetermined known quality, or they will be obtained starting from a predetermined batch.

To this end, it may be possible to select a predetermined number of sample fruits, for example 40-80 fruits of the batch, and—for each—carry out the steps of:
collecting second data relating to the estimated volume, to the actual weight and to the amount of juice extracted therefrom;
calculating, based on the estimated volume and actual weight values, the density;
calculating, based on the values of estimated volume and/or actual weight and of the amount of juice extracted, a value relating to the amount of juice extracted, preferably the percentage by weight with respect to the actual weight of the fruit;
associating—with the calculated density—the calculated value relating to the extracted amount of juice to create a pair of calibration data.

The actual weight and the estimated volume of the sample fruits can be obtained as described above, while the juice can be obtained by means of appropriate extraction means of the known type, for example a professional citrus squeezer of the known type, suitable to extract the juice from the fruit by squeezing it against a rotating shaped pin, and then measured using measuring means which vary depending on whether the value to be obtained is linked to the weight, to the volume or to a different parameter of the extracted juice.

In the preferred but not exclusive embodiment of the invention, in which the value relating to the estimated amount of juice of the fruit is the percentage by weight with respect to the weight of the fruit, the measuring means may consist of a scale for weighing the extracted juice.

The method according to the invention may therefore provide for the steps of:
collecting third data relating to all pairs of calibration data relating to sample juice fruits;
calculating the linear regression of this calibration data to obtain the reference data of the batch from which the sample fruits were taken.

The above allows to determine the juice content of any batch of juice fruits in a simple, practical and precise manner, minimising the waste of fruits.

The method is extremely easy to implement, even on existing processing lines.

This method may be useful, for example, for classifying juice fruits depending on the estimated juice content, for example classifying citrus fruits depending on whether the juice content is greater or lesser than the aforementioned EC standard.

In a further aspect of the invention, there may be provided for a computer program for implementing the aforementioned method, as well as a computer system that runs such computer program and an assembly including such computer system.

In a further aspect of the invention, there may be provided for the use of this assembly for the quality classification of juice fruits based on the estimated juice content.

The dependent claims define advantageous embodiments of the invention.

### BRIEF DESCRIPTION OF THE DRAWINGS

Further characteristics and advantages of the invention will be more apparent in light of the detailed description of some preferred but non-exclusive embodiments of the invention, illustrated by way of non-limiting example with reference to the attached drawings, wherein:

FIG. 1 is a schematic view of the orange classification process F;

FIG. 2 is a schematic view of the operation of the computer system 50;

FIG. 3 is a schematic view of the process for calibrating the reference curve relating to oranges F belonging to a batch LF.

### DETAILED DESCRIPTION OF SOME PREFERRED EMBODIMENTS

With reference to the mentioned figures, herein described is an assembly 1 for the non-destructive determination of the juice content, for example oranges F. Although hereinafter reference will be made to the latter for the sake of simplicity, it is clear that the juice fruits may vary without departing from the scope of protection of the attached claims.

The assembly 1 may comprise a machine 5 with an inlet 10 and an outlet 40 for oranges F. Although hereinafter reference will be made to the latter for the sake of simplicity, it is clear that the number of inlets and outlets may vary without departing from the scope of protection of the attached claims.

Scanning means 20, which may be made as disclosed by the document U.S. Pat. No. 5,449,911, and weighing means 30, for example a load cell, may be interposed between the inlet 10 and the outlet 40.

In a per se known manner, the scanning means 20 may determine the estimated volume VsF of the oranges F, while the weighing means 30 may determine the actual weight PrF thereof.

It is clear that even if the present document discloses a machine which includes both scanning and weighing means, the latter may be separated from each other without departing from the scope of protection of the attached claims.

The assembly 1 may further comprise a computer system 50, which may be operatively connected to the scanning means 20 and to the weighing means 30 to collect the data relating to the estimated volume VsF and to the actual weight PrF of the oranges F.

The operative connection may vary, for example of the physical type wired or through WiFi or similar networks. The operative connection may also be indirect, for example the data of the scanning and weighing means may be stored in a physical unit or cloud which in turn can be operatively connected to the computer system 50.

Essentially, the computer system may include a data collection unit 51 and a microprocessor unit 52, which—in a preferred but non-exclusive embodiment—may be contained in a single PLC unit 53.

However, it is clear that even if the present document discloses a single PLC unit which includes both the data collection unit 51 and the microprocessor unit 52, the latter may be separated from each other without departing from the scope of protection of the attached claims. For example, the data may be stored in one or more databases in the cloud, and the microprocessor unit may be a PC or a laptop which can be connected to the cloud.

The data collection unit 51 may collect the data relating to the estimated volume VsF and the actual weight PrF of oranges F in any manner, whether manual or automatic. Besides this data, the data collection unit 51 may collect one or more plurality of pairs of reference data Dr1, % r1; Dr2, % r2; Dr3, % r3 . . . regarding juice fruits.

The reference data may be present on a storage unit of the PLC 53 or it may be loaded from a cloud.

Suitably, each pair of reference data may consist of a reference density value Dr1; Dr2; Dr3 . . . and a corresponding value relating to a reference amount of juice % r1; % r2; % r3 . . . , for example, the percentage by weight of juice present in the fruit.

It is clear that even if the present document discloses a value relating to an amount of juice as the percentage by weight of juice present in a fruit, this value may vary without departing from the scope of protection of the attached claims.

Basically, the set of this data represents a curve having—in abscissa or ordinate—the density values and—in abscissa or ordinate—the percentage values of juice.

Such curve may be predetermined based on the characteristics of the fruit, for example quality and source, or it may be calibrated using the procedure described hereinafter

The microprocessor unit 52 may run a computer program, whether resident or cloud-based, which may calculate the density DcF of the orange F starting from the estimated volume and actual weight VsF, PrF data mentioned above and estimate the juice percentage % sF of the orange F by means of the aforementioned curve.

This allows to estimate—with reasonable certainty—the juice content of each orange F without damaging it.

The estimated juice percentage % sF of the orange F, as observed above, may be used for classifying oranges from a quality point of view. For example, according to the aforementioned EC standard, the oranges may be marketed fresh or not fresh depending on whether the estimated juice percentage % sF of the orange F is greater or lesser than 30%.

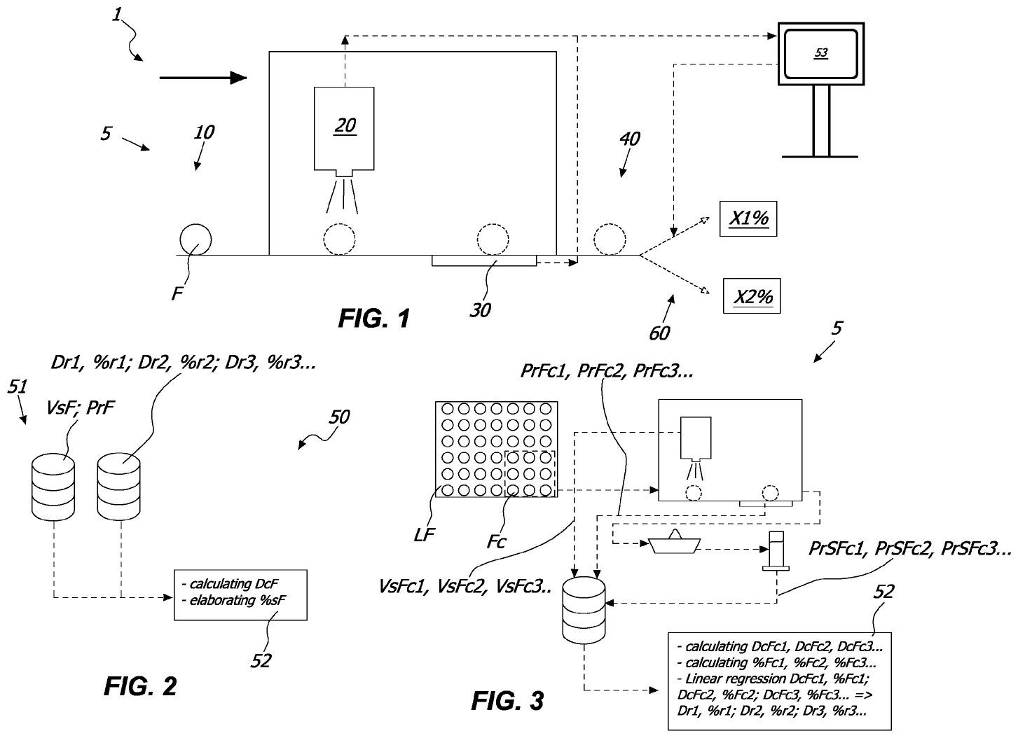

The selection may be carried out manually or, as illustrated in FIG. 1, automatically. In this case, the computer system 50 and means 60 for automatically diverting the orange F toward two or more containers X1%, X2% designed to contain oranges classified differently, for example oranges according to the EC standard mentioned, must be operatively connected directly or indirectly.

Should the reference data Dr1, % r1; Dr2, % r2; Dr3, % r3 . . . be unknown, or in any case should one seek reference data relating to a predetermined batch of oranges LF, 40-80 sample oranges Fc1, Fc2, Fc3 . . . may be taken from this batch and made to pass once or several times through scanning means 20 and weighing means 30.

This will allow to obtain the estimated volume VsFc1, VsFc2, VsFc3 . . . and actual weight PrFc1, PrFc2, PrFc3 . . . values of the sample oranges Fc.

Then, the juice extracted using appropriate extraction means 70 may be extracted from each of the latter. The extracted juice may be then weighed to obtain the weight PrSFc1, PrSFc2, PrSFc3 . . . .

Then, the microprocessor unit 52 may run a sub-program for calculating—based on the aforementioned data—the density value DcFc1, DcFc2, DcFc3 . . . and the juice percentage % Fc1, % Fc2, % Fc3 . . . relating to each sample Fc.

This will allow to create a plurality of calibration data DcFc1, % Fc1; DcFc2, % Fc2; DcFc3, % Fc3 . . . consisting of the calculated density pairs DcFc1, DcFc2, DcFc3 . . . and percentage of juice % Fc1, % Fc2, % Fc3 . . . extracted for each sample-orange Fc.

These data may be collected in the database 54, and taken from here by the microprocessor unit 52 and used to calculate the linear regression thereof, so as to obtain the reference data Dr1, % r1; Dr2, % r2; Dr3, % r3 . . . relating to the batch LF.

Therefore, the operations above may be repeated for each of the remaining oranges of this batch for example with the aim of classifying them according to the aforementioned EC standard.

Possibly, the microprocessor unit 52 may be configured so that—when determining the estimated volume VsFc1, VsFc2, VsFc3 . . . a nd the actual weight PrFc1, PrFc2, PrFc3 . . . of the sample oranges Fc—it discards the data relating to oranges having characteristics already measured previously. In this manner, the entire calibration process and subsequent sorting of the fruits will be fully automated, optimising work times and making the procedure easier to reproduce and objectively repeatable, eliminating the subjectivity of the measurements carried out manually.

In the light of the above, it is clear that the invention attains the pre-set objectives. The invention is susceptible to numerous modifications and variants all falling within the inventive concept outlined in the attached claims. All details can be replaced by other technically equivalent elements without departing from the scope of protection of the invention.

Even though the invention has been described with particular reference to the attached figures, the reference numerals used in the description and in the claims are meant for improving the intelligibility of the invention and thus do not limit the claimed scope of protection in any manner whatsoever.

The invention claimed is:

1. A computer-implemented method for a non-destructive determination of a juice content of at least one juice fruit, comprising at least the following steps:
collecting first data relating to an estimated volume (VsF) and an actual weight (PrF) of said at least one juice fruit (F);
collecting at least one first plurality of pairs of reference data (Dr1, % r1; Dr2, % r2; Dr3, % r3 . . . ) for said at least one juice fruit, each pair of reference data consisting of a reference density value (Dr1; Dr2; Dr3 . . . ) and a corresponding value relating to a reference amount of juice (% r1; % r2; % r3 . . . );
calculating, based on said first data (VsF, PrF), a density (DcF) of said at least one juice fruit (F); and
processing, based on said at least one first plurality (52), pairs of reference data (Dr1, % r1; Dr2, % r2; Dr3, % r3 . . . ) and on said calculated density (DcF), a value relating to an estimated amount of juice (% sF) for said at least one of juice fruit (F).

2. The computer-implemented method according to claim 1, wherein said at least one juice fruit (F) is of a predetermined known quality, said at least one first plurality of pairs of reference data (Dr1, % r1; Dr2, % r2; Dr3, % r3 . . . ) relating to said predetermined known quality.

3. The computer-implemented method according to claim 1, wherein the computer-implemented method is configured to determine the juice content of juice fruits (F) belonging to a predetermined batch (LF), said reference data (Dr1, % r1; Dr2, % r2; Dr3, % r3 . . . ) being determined starting from a predetermined number of sample juice fruits (Fc1, Fc2, Fc3 . . . ) belonging to said predetermined batch (LF), the method further comprising, for each of said sample juice fruits (Fc1, Fc2, Fc3 . . . ), at least the following steps:
collecting second data relating to an estimated volume (VsFc1, VsFc2, VsFc3 . . . ), to an actual weight (PrFc1, PrFc2, PrFc3 . . . ) and to an amount of juice (PrSFc1, PrSFc2, PrSFc3 . . . ) extracted therefrom;
calculating, based on said estimated volume (VsFc1, VsFc2, VsFc3 . . . ) and actual weight (PrSFc1, PrSFc2, PrSFc3 . . . ), a density value (DcFc1, DcFc2, DcFc3 . . . ); and
calculating, based on said estimated volume (VsFc1, VsFc2, VsFc3 . . . ) and/or actual weight (PrSFc1, PrSFc2, PrSFc3 . . . ) and extracted amount of juice of juice (PrSFc1, PrSFc2, PrSFc3 . . . ), a value relating to the extracted amount of juice (% Fc1, % Fc2, % Fc3 . . . ).

4. The computer-implemented method according to claim 3, further comprising the following steps:
collecting third data relating to all pairs of data (DcFc1, % Fc1; DcFc2, % Fc2; DcFc3, % Fc3 . . . ) consisting of the calculated density (DcFc1, DcFc2, DcFc3 . . . ) and the calculated value relating to the extracted amount of juice (% Fc1, % Fc2, % Fc3 . . . ) relating to said predetermined number of sample juice fruits (Fc1, Fc2, Fc3 . . . ); and
calculating the linear regression of said pairs of calibration data (DcFc1, % Fc1; DcFc2, % Fc2; DcFc3, % Fc3 . . . ) to obtain said at least one first plurality (52) of pairs of reference data (Dr1, % r1; Dr2, % r2; Dr3, % r3 . . . ) for said predetermined batch (LF).

5. The computer-implemented method according to claim 3, wherein the predetermined number of sample juice fruits (Fc1, Fc2, Fc3 . . . ) is comprised between 40 and 80.

6. The computer-implemented method according to claim 3, wherein said value relating to an estimated amount of juice (% sF) for said at least one juice fruit (F) and/or said calculated value relating to the extracted amount of juice (% Fc1, % Fc2, % Fc3 . . . ) is an estimated by weight percentage value of the juice present in said at least one juice fruit (F) with respect to its actual weight (PrF) and/or the actual weight percentage value of juice extracted from each of the sample juice fruits (Fc1, Fc2, Fc3 . . . ) with respect to its actual weight (PrSFc1, PrSFc2, PrSFc3 . . . ).

7. A computer program for a non-destructive determination of juice content of at least one juice fruit, comprising:
first instructions to perform the computer-implemented method according to claim 4; and
second instructions which, when run by a processor, command the processor to carry out at least the steps of the computer-implemented method of calculating the density (DcF) and processing a value relating to the estimated amount of juice (% sF) for said at least one juice fruit (F).

8. The computer program according to claim 7, further comprising third instructions which, when run by the processor, command the processor to further carry out the steps of:
for each sample juice fruit (Fc1, Fc2, Fc3 . . . ), calculating the density value (DcFc1, DcFc2, DcFc3 . . . ), the value relating to the extracted amount of juice (% Fc1, % Fc2, % Fc3 . . . ) and an association thereof, so as to create a plurality of pairs of calibration data (DcFc1, % Fc1; DcFc2, % Fc2; DcFc3, % Fc3 . . . ); and
calculating a linear regression of the pairs of calibration data (DcFc1, % Fc1; DcFc2, % Fc2; DcFc3, % Fc3 . . . ) to obtain said at least one first plurality (52) of pairs of reference data (Dr1, % r1; Dr2, % r2; Dr3, % r3 . . . ) for said predetermined batch (LF).

9. A computer system for the non-destructive determination of juice content of at least one juice fruit, comprising:
a data collection unit configured to collect at least the following data:
first data relating to an estimated volume (VsF) and an actual weight (PrF) of said at least one juice fruit (F);
at least one first plurality of pairs of reference data (Dr1, % r1; Dr2, % r2; Dr3, % r3 . . . ) for said at least one juice fruit, each pair of reference data consisting of a reference density value (Dr1; Dr2; Dr3 . . . ) and a corresponding value relating to a reference amount of juice (% r1; % r2; % r3 . . . ); and
a microprocessor unit operatively connected or connectable to said data collection unit;
wherein said microprocessor unit is configured to run a computer program according to claim 8.

10.-11. (canceled)
